(19) United States
(12) Patent Application Publication
(10) Pub. No.: US 2021/0369298 A1
(43) Pub. Date: Dec. 2, 2021

(54) DUAL LUMEN DILATOR FOR USE IN TRANSSEPTAL PUNCTURES

(71) Applicant: 510 KARDIAC DEVICES, INC., Mableton, GA (US)

(72) Inventors: Jaime Eduardo Sarabia, Mableton, GA (US); David Scott Lim, Keswick, VA (US)

(21) Appl. No.: 17/404,212

(22) Filed: Aug. 17, 2021

Related U.S. Application Data
(63) Continuation-in-part of application No. 15/711,597, filed on Sep. 21, 2017, now Pat. No. 11,103,276.
(60) Provisional application No. 62/416,365, filed on Nov. 2, 2016.

(51) Int. Cl. A61B 17/34; A61B 17/00

(52) U.S. Cl. CPC A61B 17/3403; A61B 2017/00247; A61B 17/3415; A61B 17/00234

(57) ABSTRACT

A dilator assembly comprising an elongated body having a proximal end and a distal end, a first lumen extending along the body, a second lumen extending along the body so that it is a parallel to the first lumen, and a joint lumen disposed adjacent the distal end of the body, wherein both the first lumen and second lumen are in fluid communication with the joint lumen. A guidewire has a first end and a second end, and the first end of the guidewire is slidably received in the first lumen and the second end of the guidewire is slidably received in the second lumen.

### CROSS-REFERENCE TO RELATED APPLICATIONS

This application is a continuation of U.S. patent application Ser. No. 15/711,597 filed on Sep. 21, 2017, now U.S. Pat. No. 11,103,276, which claims the benefit of U.S. Provisional Patent Application No. 62/416,365 filed Nov. 2, 2016, which applications are hereby incorporated by reference in their entirety.

### FIELD OF THE INVENTION

The present disclosure relates generally to medical devices and, more specifically, to trocars and dilators/obturators.

### BACKGROUND

Increasingly, minimally invasive, catheter-based therapies are being developed that allow physicians to provide therapies to patients whose existing comorbidities may preclude them from having a needed, but more invasive, surgical procedure. Over the last 30-plus years, catheter-based procedures that involve puncturing/crossing the interatrial septum, such as cardiac ablation and balloon valvuloplasty have become commonplace. In the last 5 to 10 years, new structural heart procedures, such as transcatheter valve repair/replacement, and left atrial appendage occlusion, have gained regulatory approvals and have become increasingly common procedures performed in the cardiac catheterization laboratory or hybrid operating room. With the advent of these technologies has come an increase in the need for structural heart interventionalists (specialty physicians who perform these types of procedures) to engage and cross the interatrial septum in the heart.

Historically, crossing the septum has been the purview of pediatric cardiologists or electrophysiologists due to the prevalence of cardiac ablation procedures which require crossing the interatrial septum. However, interventional cardiologists are increasingly starting to provide therapy to the left side of the heart and the requirement to puncture the interatrial septum and provide these new therapies is increasing. Unfortunately, many of these interventional cardiologists do not perform a transseptal puncture with enough regularity to become proficient at it. For these left-sided procedures, safely puncturing the interatrial septum and gaining access to the left side of the heart is not enough. These new technologies demand a very specific and safe location when crossing the interatrial septum. Additionally, crossing the interatrial septum has been historically guided by fluoroscopy (X-ray), and more recently by echocardiographic ultrasound (intracardiac echocardiography, transephogeal echocardiography or transthoracic echocardiography). Fluoroscopy is limited in its role due to its limited ability to image soft tissue, such as the interatrial septum. Therefore, echocardiography is increasingly being relied upon to guide these types of procedures.

As such, it is desirable to provide these newly evolving structural heart interventionalists with a tool to help them safely and accurately cross the interatrial septum. For example, it is desirable to have a tool this is configured to be used in conjunction with a steerable sheath to permit the use of a back end of a standard guidewire to puncture the interatrial septum rather than using a needle. A double lumen design will help prevent the need to use multiple needles/guidewires as a single guidewire may now perform all the necessary functions.

The present invention recognizes and addresses considerations of prior art constructions and methods.

### SUMMARY

One embodiment of a dilator assembly in accordance with the present disclosure includes an elongated body having a proximal end and distal end, a first lumen extending along the body, a second lumen extending along the body so that it is a parallel to the first lumen, and a joint lumen disposed adjacent the distal end of the body, wherein both the first lumen and second lumen are in fluid communication with the joint lumen. A guidewire has first end and second end, and the first end of the guidewire is slidably received in the first lumen and the second lumen end of the guidewire is slidably received in the second lumen.

Another embodiment of a dilator assembly for use with a guidewire having a first end and a second end, include; an elongated body having a proximal end and distal end, a first lumen extending along the body, a second lumen extending along the body so that it is a parallel to the first lumen, and a joint lumen disposed adjacent the distal end of the body, wherein both the first lumen and second lumen in fluid communication with the joint lumen. A first luer has a first luer line that is fluid communication with the first lumen, and a second luer has a second luer line that is in fluid communication with the second lumen, wherein the first end of the guidewire is slidably received in both the first luer line and the first lumen and the second end of the guidewire is slidably received in both the second luer line and the second lumen.

Another embodiment of an introducer sheath assembly in accordance with the present disclosure includes a handle portion with a front end and a rear end, a introducer sheath extending outwardly from the front end of the handle portion, the introducer sheath including a device lumen configured to slidably receive a corresponding device, and a device locking assembly disposed at the rear end of the housing portion, including an elongated stem defining an axially extending bore that is confirmed to slidably receive the corresponding device, the elongated stem being axially movable with respect to the handle portion, a clamp disposed on the distal end of the elongated stem, the clamp being positionable between a locked position in which the corresponding device is axially fixed with respect to the elongated stem and an un-locked position in which the corresponding device is slidable within the axially extending bore of the elongated stem.

### BRIEF DESCRIPTION OF THE DRAWINGS

The accompanying drawings, which are incorporated in and constitute a part of this specification, illustrate one or more embodiments of the invention and, together with the description, serve to explain the principles of the invention.

A full and enabling disclosure of the present invention, including the best mode thereof, directed to one of ordinary skill in the art, is set forth in the specification, which makes reference to the appended drawings, in which:

FIG. 1 is a perspective view of a dual lumen dilator assembly in accordance with an embodiment of the present disclosure;

FIG. 2 is a partial, cross-sectional view of a distal end of the dilator assembly shown in FIG. 1, showing the two lumens combining into a simple lumen;

FIG. 3 is a partial perspective view of a central hub of the dilator assembly shown in FIG. 1, which shows details of how the two independent lumens are attached to the central hub so that they may extend along a body of the dilator assembly in parallel fashion;

FIG. 4 is a perspective view of the dilator assembly shown in FIG. 1, wherein a floppy distal end of a corresponding guidewire extends outwardly from the distal end of the dilator assembly; and

FIG. 5 is a perspective view of the dilator assembly shown in FIG. 1, wherein a stiff end of the corresponding guidewire extends outwardly from the distal end of the dilator assembly.

Repeat use of reference characters in the present specification and drawings is intended to represent same or analogous features or elements of the invention according to the disclosure.

### DETAILED DESCRIPTION OF THE PREFERRED EMBODIMENTS

Reference will now be made in detail to presently preferred embodiments of the invention, one or more examples of which are illustrated in the accompanying drawings. Each example is provided by way of explanation, not limitation, of the invention. In fact, it will be apparent to those skilled in the art that modifications and variations can be made in the present invention without departing from the scope and spirit thereof. For instance, features illustrated or described as part of one embodiment may be used on another embodiment to yield a still further embodiment. Thus, it is intended that the present invention covers such modifications and variations as come within the scope of the appended claims and their equivalents. As well, directions given for deflection of the distal portion of the introducer sheath of the present invention are given as left or right of a vertical plane that passes through longitudinal center axis of the disclosed introducer sheath assembly, when the introducer sheath is fully extended. Note, the introducer sheath assembly is, preferably, substantially symmetrical about the vertical plane.

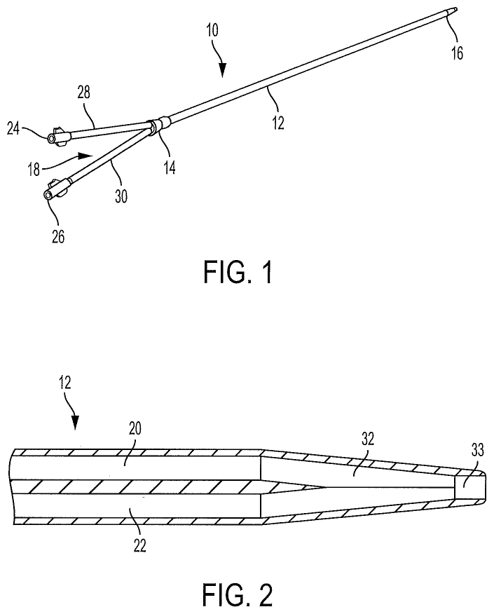

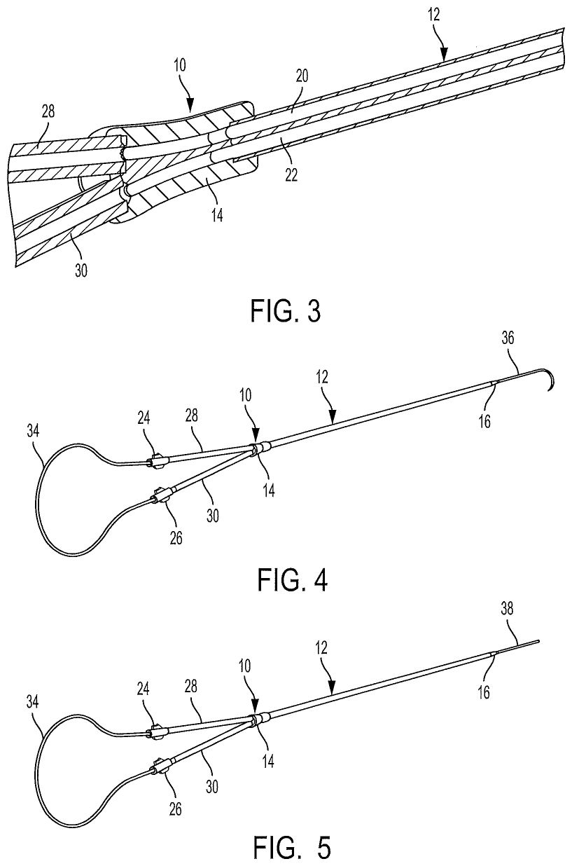

Referring now to FIGS. 1 through 3, a dual lumen dilator assembly 10 in accordance with an embodiment of the present disclosure includes an elongated body portion, a central hub 14, and first and second luer lines 28 and 30, having first and second luers 24 and 26, respectively. As been seen in FIG. 3, elongated body 12 extends outwardly from a first end of central hub 14, and a first lumen and a second lumen 20 and 22, respectively, extend along body 12 from central hub 14 to a tapered distal end 16 of body 12. First and second lumens 20 and 22 are parallel to each other and remain separated from each other until coming together at joint lumen 32, ultimately forming a single exit lumen 33, which is preferably at the distal end 16 of elongated body 12. Note, however, first and second lumens 20 and 22 may come together to form the joint lumen at other positions along elongated body 12.

Referring specifically to FIG. 2, an enlarged, cross-sectional view of distal end 16 of elongated body 12 is shown. As shown, first and second lumens 20 and 22 are parallel to each other along body 12, but combine at a joint lumen portion 32 to form a single exit lumen 33 at distal end 16 of the assembly. The integration of first and second lumens 20 and 22 may be from an extrusion or may be separate components that are manufactured independently and then joined to the extrusion for the purpose of uniting the two lumens. The geometry of joint lumen portion 32 is designed in such a manner that regardless of guidewire 34 (FIGS. 4 and 5) advancement or retraction, guidewire 34 successfully finds a lumen through which it can transverse.

Referring now to FIG. 3, a magnified and sectional view of the central hub 14 is shown. The central hub 14 diverts first and second lumens 20 and 22 from elongated body 12 of the dilator assembly away from each other and couples each lumen of the assembly to a corresponding one of luer lines 28 and 30.

Referring now to FIG. 4, a perspective view of dilator assembly 10 is shown with a guidewire 34 inserted in a first configuration. In the first configuration, a floppy, distal (front) end 36 of guidewire 34 extends outwardly from distal end 16 of body 12, as discussed in greater detail below. Referring to FIG. 5, a perspective view of dilator assembly 10 is shown with guidewire 34 inserted in a second configuration. In the second configuration, a straight, rigid proximal (back) end of guidewire 34 extends outwardly from distal end 16 and elongated body 12, as discussed in greater details below.

As noted above, dilator assembly 10 preferably includes two lumens 20 and 22 running along most of the length of the body of dilator assembly. The first and second lumens 20 and 22 combine at joint lumen portion 32 somewhere along the length of the dilator (preferably towards the distal end of the elongated body of the dilator assembly) to form a single exit lumen 33. This configuration of dilator assembly 10 allows a physician to track the dilator/sheath assembly over a previously placed guidewire 34 (FIG. 4), as is the normal practice. Once the desired location of their transseptal dilator assembly system is attained, the proximal (back) end 38 of the guidewire is inserted into the second, open lumen 22 by way of second luer 26 and second luer line 30 and advanced towards distal end 16 of body 12. The floppy, distal (front) end 36 of the guidewire is then retracted, which opens up the exit lumen 33 for advance of the stiff, proximal (back) end 38 of the guidewire. The stiff back end 38 of the guidewire is then used to puncture the interatrial septum. Upon puncturing the septum, the stiff back end 38 of the guidewire is retracted, the dilator assembly 10 is advanced into the left atrium, and then the floppy front end 36 of the guidewire is re-advanced into the left atrial via exit lumen 33, thus providing safe, guidewire access into the left atrium. These operations occur without requiring the removal or reinsertion of multiple guidewires and needles, thus increasing the efficiency and safety of transseptal punctures.

Dilator assembly 10 negates the need to use a separate needle to puncture the interatrial septum and, therefore, provides time savings as it eliminates the need for device exchanges. When the floppy, distal end 36 of the guidewire is needed for dilator assembly advancement and tracking, it is extended out of the distal end of the dilator assembly's body and tracked over as needed for the advancement of the dilator assembly. Once the functionality of tracking and advancement is no longer required, distal end 36 end of the guidewire is retracted within body 12 of dilator assembly 10 and the more rigid proximal end 38 of the guidewire is used for puncture. These two features of the guidewire are used interchangeably and rapidly as needed.

While one or more preferred embodiments of the invention are described above, it should be appreciated by those skilled in the art that various modifications and variations can be made in the present invention without departing from the scope and spirit thereof. It is intended that the present invention cover such modifications and variations as come within the scope and spirit of the appended claims and their equivalents.

1. Method for puncturing a septum wall, said method comprising:
providing a dilator assembly comprising:
an elongated body having a proximal end and a distal end, a first lumen extending along the body, a second lumen extending along the body so that it is a parallel to the first lumen, and a joint lumen disposed adjacent the distal end of the body, and
a guidewire having a first end and a second end, inserting said first end of said guidewire into said first lumen at said proximal end, inserting said second end of said guidewire into said second lumen at said proximal end, advancing said second end of said guidewire out of said distal end, and puncturing said septum wall using said second end of said guidewire.

2. The method of claim 1, comprising retracting said second end of said guidewire into said second lumen.

3. The method of claim 1, comprising advancing the dilator assembly through the puncture to dilate the puncture.

4. The method of claim 1, comprising advancing said first end for a guidewire access to a left atrium.

5. The method of claim 1, comprising before advancing the second end, advancing said first end of said guidewire through the joint lumen for guiding said dilator to a puncture site in said septum, and retracting said first end of said guidewire into said first lumen.

6. The method of claim 1, wherein puncturing said septum wall, is puncturing an interatrial septum wall.

7. The method of claim 1, wherein providing a dilator assembly comprises providing said dilator assembly with a joint lumen having a single exit lumen.

8. The method of claim 1, not requiring removal or reinsertion of multiple guidewires or needles.

9. The method of claim 1, wherein puncturing of said septum wall can be performed without using a separate needle.

10. The method of claim 1, wherein providing the guidewire comprises providing a guidewire wherein said first end of said guidewire has a first rigidity for advancement and tracking of said dilator assembly.

11. The method of claim 1, wherein providing the guidewire comprises providing a guidewire wherein the second end has a second rigidity being more rigid than the first rigidity.

12. The method of claim 1, comprising interchangeably advancing and retracting either said first end or said second end of said guidewire out of the distal end of said elongate body.

13. A method for puncturing a septum wall, said method comprising:
interchangeably advancing and retracting a first end of a guidewire and a second end of said guidewire out of a distal end of a dilator assembly for puncturing a septum wall.

14. The method of claim 13, comprising interchangeably advancing and retracting a first end of a guidewire and a second end of said guidewire out of a single exit lumen at said distal end of said dilator assembly.

15. A method for puncturing a septum wall, said method comprising:
inserting a first end of a guidewire into a first lumen at a proximal end of a dilator assembly,
inserting a second end of said guidewire into a second lumen at said proximal end of said dilator assembly,
advancing said second end of said guidewire out of a distal end of said dilator assembly, and
puncturing said septum wall using said second end of said guidewire.
